(19) United States
(12) Patent Application Publication
(10) Pub. No.: US 2007/0031818 A1
(43) Pub. Date: Feb. 8, 2007

(54) Assay for distinguishing live and dead cells

(75) Inventors: Vadim Kutsyy, Cupertino, CA (US); Jinhong Fan, San Mateo, CA (US); Eugeni A. Vaisberg, Foster City, CA (US)

Correspondence Address: BEYER WEAVER & THOMAS, LLP, P.O. BOX 70250, OAKLAND, CA, 94612-0250, US

(73) Assignee: Cytokinetics, Inc., A Delaware Corporation

(21) Appl. No.: 11/355,258

(22) Filed: Feb. 14, 2006

Related U.S. Application Data
(63) Continuation-in-part of application No. 11/082,241, filed on Mar. 15, 2005.
(60) Provisional application No. 60/588,907, filed on Jul. 15, 2004.

(51) Int. Cl. C12Q 1/00; C12Q 1/68; G06K 9/00

(52) U.S. Cl. 435/4; 382/128; 435/6

(57) ABSTRACT

Image analysis methods and apparatus are used for distinguishing live and dead cells. The methods may involve segmenting an image to identify the region(s) occupied by one or more cells and determining the presence of a particular live-dead indicator feature within the region(s). In certain embodiments, the indicator feature is a cytoskeletal component such as tubulin. In certain embodiments, the methods may involve determining the value of an indicator expression that is based on cellular components such as DNA and/or cellular protein. Prior to producing an image for analysis, cells may be treated with a marker that highlights the live-dead indicator in the image.

### CROSS REFERENCE TO RELATED APPLCATIONS

This application is a continuation-in-part of U.S. patent application Ser. No. 11/082,241, filed Mar. 15, 2005, titled ASSAY FOR DISTINGUISHING LIVE AND DEAD CELLS which claims priority under 35 USC § 119(e) from U.S. Provisional Patent Application No. 60/588,907, filed Jul. 15, 2004 and titled ASSAY TO DISTINGUISH LIVE AND DEAD CELLS. This application is also related to the following US Patent documents: U.S. patent application Ser. No. 09/729,754, filed Dec. 4, 2000, titled CLASSIFYING CELLS BASED ON INFORMATION CONTAINED IN CELL IMAGES; U.S. patent application Ser. No. 09/792,013, filed Feb. 20, 2001 (Publication No. US-2002-0154798-A1), titled EXTRACTING SHAPE INFORMATION CONTAINED IN CELL IMAGES; and U.S. patent application Ser. No. 10/719,988, filed Nov. 20, 2003 (Publication No. US-2005-0014217-A1), titled PREDICTING HEPATOTOXICITY USING CELL BASED ASSAYS. Each of the references listed in this section is incorporated herein by reference in its entirety and for all purposes.

Methods, computer program products, and apparatus for image analysis of biological cells are provided. In certain embodiments, methods, computer program products, and apparatus for automatically analyzing images to determine whether individual cells within those images are alive or dead.

A number of methods exist for investigating the effect of a treatment or a potential treatment, such as administering a drug or pharmaceutical to an organism. Some methods investigate how a treatment affects the organism at the cellular level so as to determine the mechanism of action by which the treatment affects the organism. One approach to assessing effects at a cellular level involves capturing images of cells that have been subjected to a treatment. At times, it will be desirable to determine whether individual cells within a population of cells were alive or dead during image capture. For example, a researcher may need to quickly determine the relative numbers of live and dead cells in a population treated with a chemical compound or other stimulus. This may show the effectiveness of a treatment on pathogenic cells or the potential side effects of the treatment on benign cells.

Further, in some lines of research, phenotypic characteristics of dead cells may mask interesting morphological characteristics resulting from a treatment under investigation. Techniques that distinguish live and dead cells could unmask the effect by allowing researchers to focus on live cells and thereby determine the true impact of the treatment on live cells. Such techniques could also prevent researchers from mistakenly concluding that a general morphological feature of dead cells is a specific result of the treatment under investigation.

What is needed therefore is an improved image analysis technique for distinguishing live cells from dead cells.

Image analysis methods and apparatus for distinguishing live and dead cells are described herein. These may involve segmenting an image to identify the region(s) of the image occupied by one or more cells and determining the presence or quantity of a particular live-dead indicator feature within the region(s). In some embodiments, the indicator feature is a cytoskeletal component such as tubulin. In other embodiments, different cellular components such as DNA and/or non-specific cellular protein may serve this purpose. Prior to producing an image for analysis, cells may be fixed and treated with a marker that highlights the live-dead indicator in the image. In the case of tubulin, the marker will co-locate with tubulin and provide a signal that is captured in the image (e.g., a fluorescent emission). Similarly, markers that co-locate with DNA and/or all cellular proteins may be used to provide signals.

One method of distinguishing live cells from dead cells in a population of cells comprises (a) providing one or more images of the population of cells; (b) automatically analyzing the image; and (c) automatically classifying at least one cell in the population of cells as live or dead.

In certain embodiments, automatically analyzing the image comprises analyzing one or more cytoskeletal components in at least one cell in the population of cells. In certain embodiments, analyzing one or more cytoskeletal components comprises determining the presence or absence of the one or more cytoskeletal components. In certain embodiments, analyzing one or more cytoskeletal components comprises determining the concentration of the one or more cytoskeletal components. In certain embodiments, analyzing one or more cytoskeletal components comprises determining the distribution of the one or more cytoskeletal components. In certain embodiments, analyzing one or more cytoskeletal components comprises determining the intensity of one or more markers for such one or more cytoskeletal components.

In certain embodiments, the population of cells is one cell. In certain embodiments, the population of cells is more than one cell.

In certain embodiments, tubulin is the cytoskeletal component. The tubulin may exist in any form, including polymerized states such as microtubules.

In certain embodiments, automatically analyzing the image comprises analyzing one or more cellular components selected from cellular protein and/or DNA in at least one cell in the population of cells. In certain embodiments, analyzing the DNA and/or cellular protein comprises determining the concentration of the DNA and/or cellular protein. In certain embodiments, analyzing the DNA and/or cellular protein comprises determining the distribution of the DNA and/or cellular protein. In certain embodiments, analyzing the DNA and/or cellular protein comprises determining the intensity of one or more markers for the DNA and/or cellular protein.

In certain embodiments, analyzing the image comprises determining statistical properties of the intensity of a marker. For example, one or more of the mean intensity, standard deviation (square root of the second moment), skewness (third moment), and kurtosis (fourth moment) of the intensity as measured across all or part of a cell may be used to analyze the image. Such statistical properties may also be referred to as features.

In some embodiments, the method further comprises automatically segmenting the image prior to determining the information about tubulin or other cytoskeletal or cellular component or components. In certain embodiments, segmentation comprises identifying nuclei of one or more cells in the image. In certain embodiments, segmentation further comprises determining cell boundaries within the image. The cell boundaries can be determined using, for example, (i) a non-specific marker for proteins in the cell or (ii) a marker for a plasma membrane component. In certain embodiments, segementation further comprises determining nuclear and/or cytoplasm boundaries within the image.

In certain embodiments, the method further comprises (d) determining one or more morphological features of the cells in the image; and (e) determining the degree to which the one or more morphological features occurs in live cells and/or dead cells. Examples of morphological features include the overall cell shape, the structure of particular organelles such as Golgi or the nucleus, and the structure of particular cytoskeletal components.

In certain embodiments, the method is performed in a manner that allows live cells to continue functioning after treatment with a stimulus under investigation, but without any additional treatment intended to facilitate imaging of the live-dead indicator feature. Such additional treatments could, in some circumstances, interfere with the functioning of live cells and may even mask specific effects of a treatment (e.g., hide certain cellular morphological features of interest). In certain embodiments, the method further comprises exposing the population of cells to a stimulus; fixing the population of cells; and marking one or more cytoskeletal or other cellular components in the population of cells with one or more markers that is specific for the one or more cytoskeleton or other cellular components. Of course, the order be reversed; i.e., marking may be followed by fixing.

In certain embodiments, a stimulus is applied in different doses or levels to populations of cells. The phenotypic effects of the stimulus can then be determined as a function of dose or level. For at least two of the different doses or levels, the impact on live and dead cells is assessed. In certain embodiments, the method further comprises repeating steps (a)-(c) multiple times, each time for a different population of cells, such that the different populations of cells have been exposed to different doses or levels of a stimulus. The stimulus-paths of different stimuli or of different doses or levels of a stimulus can be compared to make assessments about the similarity of cellular responses to different stimuli or different doses or levels of a stimulus.

In certain embodiments, the method employs a mixture model of two distributions, one for live cells and one for dead cells. In certain embodiments, each distribution is a Gaussian distribution representing a distribution of the concentration of tubulin in a single cell (indicated by the mean intensity of a tubulin marker in the cell for example). In certain embodiments, the Gaussian distribution for the dead cells has a smaller mean than a Gaussian distribution for the live cells. In certain embodiments, each distribution is a Gaussian distribution of linear or non-linear combinations of cytoskeletal or other cellular features.

Also provided are methods of producing models for automatically distinguishing live cells from dead cells. In certain embodiments, the method comprises (a) providing one or more images of live cells and dead cells; (b) determining a level of one or more cytoskeletal components for multiple cells in the one or more images; and (c) from the levels obtained in (b), determining two Gaussian distributions for the levels of the one or more cytoskeletal components, one for live cells and one for dead cells. In certain embodiments, the levels of the one or more cytoskeletal components is a measure of the mean concentration of the one or more cytoskeletal components in a cell.

In certain embodiments, the one or more images provided in (a) include images of positive and negative control populations having relatively high percentages of dead and live cells.

In certain embodiments, the images are segemented prior to determining a level of one or more cytoskeletal components for multiple cells in one or more images by automatically identifying nuclei of individual cells in the images and/or automatically determining cell boundaries within the image.

In certain embodiments, determining two Gaussian distributions for the levels of the one or more cytoskeletal components, comprises (i) providing an empirical distribution of the level of the cytoskeletal component in individual cells, which can be visualized as a histogram of the number of cells in the images versus the level of the cytoskeletal component in an individual cell; and (ii) using this empirical distribution to determine a mixture of the two Gaussian distributions. In certain embodiments, an Expectation Maximization (EM) procedure is used to identify a mean and a standard deviation for each of the two Gaussian distributions.

In certain embodiments, the method comprises (a) providing one or more images of live cells and dead cells; (b) evaluating an indicator expression containing one or more features from cells in the one or more images to produce indicator expression values for the cells; and (c) from the indicator expression values obtained in (b), determining two Gaussian distributions for the indicator expression values, one for live cells and one for dead cells. In certain embodiments, the indicator expression contains one or more of the mean intensity of a DNA marker within the cell, one or more moments of the intensity of the DNA marker within the cell, the area of the DNA marker occupies within the cell, the mean intensity of a cellular protein marker within the cell, one or more moments of the intensity of the cellular protein marker within the cell, and the area the cellular protein marker occupies within the cell.

In certain embodiments, the one or more images provided in (a) include images of positive and negative control populations having relatively high percentages of dead and live cells.

In certain embodiments, determining two Gaussian distributions for the values of indicator expressions comprises (i) providing an empirical distribution of the values of the indicator expression in individual cells, which can be visualized as a histogram of the number of cells in the images versus the value of the indicator expression in an individual cell; and (ii) using this empirical distribution to determine a mixture of the two Gaussian distributions. In certain embodiments, an Expectation Maximization (EM) procedure is used to identify a mean and a standard deviation for each of the two Gaussian distributions.

Also provided are computer program products including machine-readable media on which are stored program instructions for implementing at least some portion of the methods described above. Any of the methods described herein may be represented, in whole or in part, as program instructions that can be provided on such computer readable media. Also provided are various combinations of data and data structures generated and/or used as described herein.

These and other features and advantages will be described in more detail below with reference to the associated figures.

### BRIEF DESCRIPTION OF THE DRAWINGS

FIG. 1 presents two images of cells marked with a marker for tubulin: a left image of a control population of cells treated with DMSO, and a right image of a test population of cells treated with the compound CCCP, which kills cells.

FIG. 2 is a flowchart depicting one method for producing a model that can be used to distinguish live and dead cells in accordance with certain embodiments.

FIG. 3A presents a pair of images in which the nuclei of individual cells in two different cell populations have been identified as part of a segmentation procedure. A DNA stain was imaged to permit identification of the nuclei.

FIG. 3B presents the images of the cell populations of FIG. 3A, but with the boundaries of the individual cells identified to complete the cell segmentation procedure. A non-specific protein stain was imaged to permit identification of the cellular boundaries.

FIG. 3C again presents the cell populations of FIG. 3A, but with cell boundaries elucidated as in FIG. 3B and with a tubulin marker highlighted to allow distinction of live and dead cells.

FIG. 4A is a histogram of mean tubulin marker intensity (per cell). The cells providing the data in this histogram were generated from a control population treated with DMSO and have relatively high percentage of live cells.

FIG. 4B is a histogram similar to that of FIG. 4A, but comprised of data taken from test cells treated with the compound CCCP, as well as control cells treated with DMSO. The histogram peak associated with dead cells is much more pronounced in FIG. 4B than in FIG. 4A.

FIG. 5 is a flowchart depicting one method for using a model to distinguish live and dead cells in accordance with certain embodiments.

FIG. 6A is a graph showing how CCCP concentration affects the total number of cells in an image, as well as the number of dead cells and the number of live cells.

FIG. 6B is a graph showing the effect of a different drug (diclofenac) on the total cell area in live cells and dead cells.

FIG. 6C is a graph showing how another drug (tacrine) impacts the mean intensity (on a cell-by-cell basis) of a marker for the TGN (trans-Golgi network) in live cells and dead cells.

FIG. 7 is a diagrammatic representation of a computer system that can be used with the methods and apparatus described herein.

FIG. 8 is a histogram of estimated log₂ (DM1α) (per cell) for two sets of detergent treated test cells and control cells. Gaussian curves generated from the data are also shown.

FIG. 9 is a histogram of log (P/1-P) (per cell) for three sets of non-detergent treated test cells and control cells. Gaussian curves generated from the data are also shown.

Tubulin and related cytoskeletal markers may serve as indicators of whether a cell is alive or dead. Other cellular components may also serve this purpose. It has been found, for example, that the total quantity of cellular protein, as indicated by particular markers, indicates whether a cell is live or dead. The amount and distribution of DNA within a cell also provides some indication of whether a cell is alive or dead. Much of the description in this application refers to cytoskeletal components, such as tubulin, as examples of indicators for determining whether a cell is alive or dead. However, the methods and other aspects described extend to other cellular components whose presence, levels, and/or distribution within a cell also correspond to live and dead cells.

Models and methods of generating such models are provided to take advantage of these discoveries. In some embodiments, the models can automatically classify a cell as either alive or dead depending upon the level of tubulin found in the cell. In certain embodiments, automated image analysis techniques are employed to identify cells in an image, determine the level of tubulin in each identified cell, and based on the level of tubulin, classify individual cells as alive or dead. In some embodiments, the models are “mixture models” comprised of two ranges of tubulin levels, a lower range indicating dead cells and an upper range indicating live cells. In a specific embodiment, each range is represented as a Gaussian distribution with its own mean and standard deviation. Methods of producing such models and methods of applying such models to sample cells to determine whether such cells are alive or dead are provided.

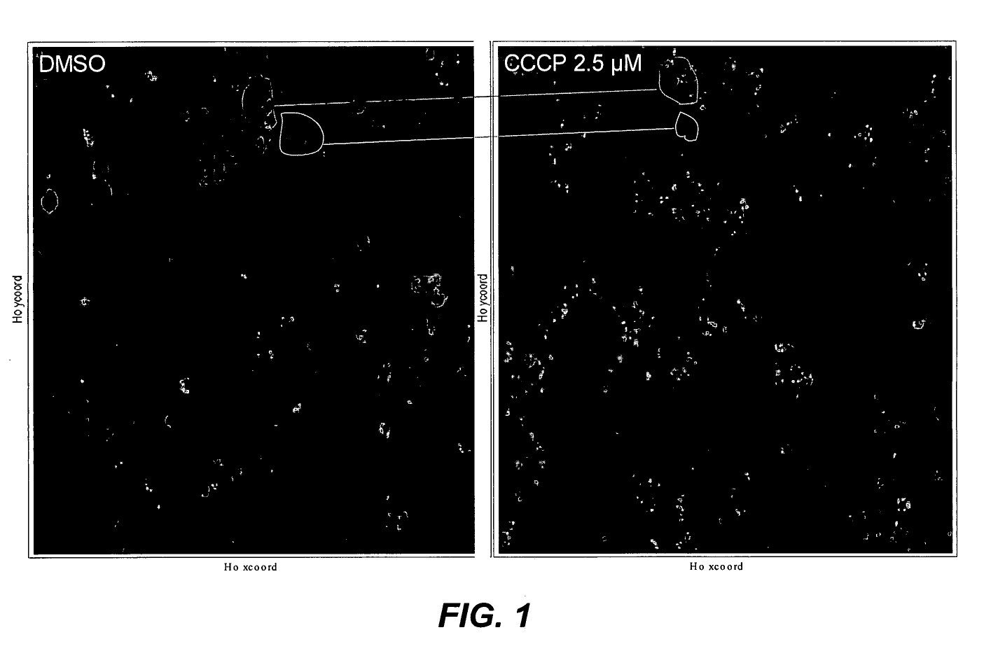

FIG. 1 presents two images of cells: a left image of a control population of cells treated with DMSO, and a right image of a test population of cells treated with the compound carbonyl cyanide 3-chlorophenylhydrazone (herein CCCP), a poison which acts on the cellular respiratory pathway. After treatment with DMSO or CCCP, the cells were fixed and stained with multiple markers. Three of these markers are shown in the image: red indicates DNA, blue indicates tubulin, and green indicates the trans-Golgi network. The figure shows that a population of cells treated with CCCP contains far fewer cells with significant tubulin concentration (as indicated by a reduction in the number of cells having a blue color in comparison to those in a control population treated with DMSO). Note that individual cells are identified (whether alive or dead) by a small red area in the central region. This red area is associated with DNA in the cell nucleus. The number of cells having a green color (for the trans-Golgi network marker) is greatly increased in the CCCP treated cells image. This is not necessarily an indication that the dead cells have increased levels of Golgi. Rather, it merely indicates that the blue tubulin intensity is not present at a level that masks the green Golgi signal.

In the context of the description provided, a cell is said to be “dead” when it ceases to carry on any significant cellular functions such as respiration, mitosis, etc. Thus, the term “dead,” as used herein, corresponds to the conventional meaning of the term. Note that this applies to cells that have died by any of the various processes that typically lead to cell death. These processes include apoptosis, necrosis, paraptosis, etc.

As indicated, a dead cell may be identified by a reduced level of tubulin in the region bounded by the cell. In certain embodiments, other cytoskeletal proteins, such as actin, may also serve as indicators of cell death. Further, the tubulin, actin, or other cytoskeletal indicator protein(s) may take various forms including microtubules, unpolymerized tubulin, actin filaments, intermediate filaments, and various other assemblies, each of which may, in certain embodiments, indicate whether a cell is alive or dead. In certain embodiments, various non-cytoskeletal proteins serve as indicators of cell death. In one example, the overall protein content of a cell, as presented by the Alexa 647 succinimidyl ester (Alexa 647), also indicates whether a cell is alive or dead. In certain embodiments, DNA is used together with overall protein to indicate whether a cell is alive or dead.

The level of tubulin or other cytoskeletal indicator in a cell may be measured as the intensity of a marker for the indicator appearing in an image of the cell. The local intensity of a tubulin marker in an image generally corresponds directly to the local tubulin concentration at particular regions within a cell. Examples of tubulin markers include fluorescently labeled antibodies to tubulin (e.g., DM1-α, YL1-2, and 3A2 antibodies), cells expressing GFP (or YFP, etc.) labeled tubulin, and the like.

In general, a marker is linked to or otherwise co-located with a cell component under investigation. It serves as a labelling agent and should be detectable in an image of the relevant cells. In other words, the location of the signal source (i.e., the location of the marker within the cells) appears in the image. The marker should provide a strong contrast to other features in a given image. To this end, the marker may be luminescent, radioactive, fluorescent, etc. Various stains and compounds may serve this purpose. Examples of such compounds include fluorescently labelled antibodies to the cellular component of interest, fluorescent intercalators, and fluorescent lectins. The antibodies may be fluorescently labeled either directly or indirectly. The labelling agent typically emits a signal at an intensity related to the concentration of the cell component to which the agent is linked. For example, the signal intensity may be directly proportional to the concentration of the underlying cell component.

In certain embodiments, the image analysis for determining whether a cell was alive or dead is used in conjunction with additional image analysis for identifying one or more other relevant morphological characteristics or biological states of the cell (that may result from treatment with a stimulus under investigation). Of course, cellular components associated with these other morphological characteristics also may be highlighted by marking. Examples of such components include proteins and peptides, lipids, polysaccharides, nucleic acids, etc. Sometimes, the relevant component will include a group of structurally or functionally related biomolecules such as micells or vesicles. Alternatively, the component may represent a portion of a biomolecule such as a polysaccharide group on a protein, or a particular subsequence of a nucleic acid or protein. In certain embodiments, sub-cellular organelles and assemblies serve as the components (e.g., the Golgi, cell nuclei, the cytoskeleton, etc.).

In certain embodiments, markers for DNA or other nuclear component (e.g., histones) are employed to facilitate segmentation. Examples of such markers include DAPI or Hoechst 33341 stains for DNA (available from Molecular Probes, Inc. of Eugene, Oreg.) and antibodies to histones such as an antibody for a phosphorylated histone, e.g., phospho-histone 3 (pH3). Another option is to use cells expressing a GFP-histone2B (or any other GFP-tagged protein that functionally co-localizes with nuclear DNA). In addition to markers for the cell nucleus, other markers can be employed facilitate identification of cells. Examples of such markers include Alexa Flour 647 available from Molecular Probes, Eugene, Oreg. (a non-specific marker for free amine groups in proteins) and markers that bind to particular proteins in the cell membrane.

As indicated above, the signal from the Alexa 647 marker may be employed, in certain embodiments, for the purpose of indicating whether a cell is alive or dead. Relatively low signal from the marker indicates that the cell is dead. Other markers for overall protein content may be employed for the same purpose in certain embodiments.

As used herein, the term “stimulus” refers to something that may influence the biological condition of a cell. Often the term is used synonymously with “agent” or “manipulation” or “treatment.” Stimuli may be materials, radiation (including all manner of electromagnetic and particle radiation), forces (including mechanical (e.g., gravitational), electrical, magnetic, and nuclear), fields, thermal energy, and the like. General examples of materials that may be used as stimuli include organic and inorganic chemical compounds, biological materials such as nucleic acids, carbohydrates, proteins and peptides, lipids, various infectious agents, mixtures of the foregoing, and the like. Other general examples of stimuli include non-ambient temperature, non-ambient pressure, acoustic energy, electromagnetic radiation of all frequencies, the lack of a particular material (e.g., the lack of oxygen as in ischemia), temporal factors, etc.

One class of stimuli is chemical compounds including compounds that are drugs or drug candidates and compounds that are present in the environment. Related stimuli involve suppression of particular targets by siRNA or other tool for preventing or inhibiting expression. The biological impact of these and other stimuli may be manifest as phenotypic changes that can be detected and characterized in accordance with embodiments described herein.

The term “image” is used herein in its conventional sense, but with notable extensions. For example, the concept of an image extends to data representing collected light intensity and/or other characteristics such as wavelength, polarization, etc. on pixel-by-pixel basis within the relevant field of view. An “image” may also include derived information such as groups of pixels deemed to belong to individual cells—as a result of segmentation. The image need not ever be visible to researchers or even displayed in a manner allowing visual inspection. In certain embodiments, computational access to the pixel data is all that is required.

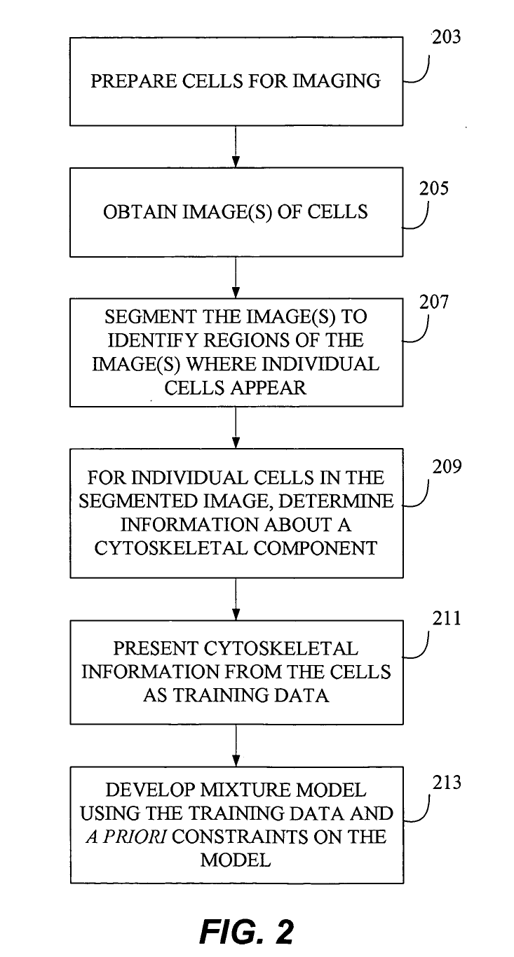

FIG. 2 presents a flowchart depicting one method for producing a model that can be used to determine whether an individual cell is live or dead. As shown in a block 203, the method begins by preparing the cell populations that are to be used for imaging. In certain embodiments, a sandwich culture is employed. In this preliminary operation, some cell populations are treated as controls (assumed to have a high fraction of cells that are alive) and other cell populations are treated as test samples (assumed to have a significant fraction of cells that are dead). Upon completion of treatment, cells are fixed and stained with appropriate markers. The test cells will all have been treated with a compound or other stimulus known to kill a significant percentage of the cells in a given population. Together, the control and test cell populations provide relatively large numbers of live and dead cells. The size of these populations should be large enough to provide a training set sufficient to generate a model that can reliably distinguish live cells from dead cells. Typically, at this stage in the process, one does not know exactly how many cells have been killed and how many remain alive (in either the control set or the test set).

As illustrated in FIG. 2, block 205, the process obtains images of the cells provided in 203. The images and imaging conditions are chosen to allow extraction of relevant features that can be used to identify individual cells and characterize them as live or dead. These images provide the raw data for a training set used to build a live-dead model. From the cellular images, the process extracts multiple cellular features, at least one of which allows segmentation of the cells and at least one of which provides a measure of the concentration of a cytoskeletal component (e.g., tubulin) or of DNA and protein content over some or all regions of the cell. In some cases a morphological indicator of interest is also taken with the image (e.g., the trans-Golgi network marker shown in FIG. 1).

In certain embodiments, the method first identifies the locations of the discrete cells in the image. This may be accomplished by segmentation. See block 207 in FIG. 2. Segmentation can be performed by various techniques including those that rely on identification of discrete nuclei and those that rely on the location of cytoplasmic proteins or cell membrane proteins. Exemplary segmentation methods are described in US Patent Publication No. US-2002-0141631-A1 of Vaisberg et al., published Oct. 3, 2002, and titled “IMAGE ANALYSIS OF THE GOLGI COMPLEX,” and US Patent Publication No. US-2002-0154798-A1 of Cong et al. published Oct. 24, 2002 and titled “EXTRACTING SHAPE INFORMATION CONTAINED IN CELL IMAGES,” both of which are incorporated herein by reference for all purposes.

In one approach, individual nuclei are first located to identify discrete cells. Any suitable stain for DNA or histones may work for this purpose (e.g., the DAPI and Hoechst stains mentioned above). Individual nuclei can be identified by performing, for example, a thresholding routine on images taken at a channel for the nuclear marker. After the nuclei are identified, cell boundaries can then be determined around each nucleus. In one embodiment, a non-specific marker for proteins such as Alexa 647 is used with an appropriate algorithm to identify cell boundaries. In another embodiment, a marker for a cell membrane protein is used for this purpose. In either case, a watershed algorithm has been found useful in determining boundaries of individual cells within the images.

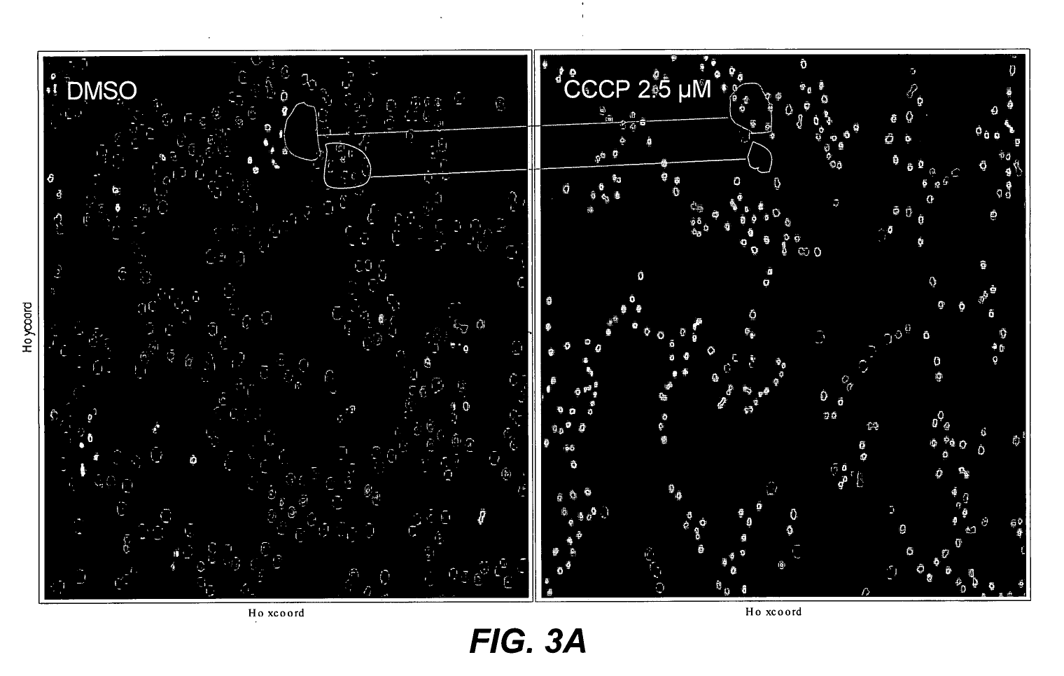

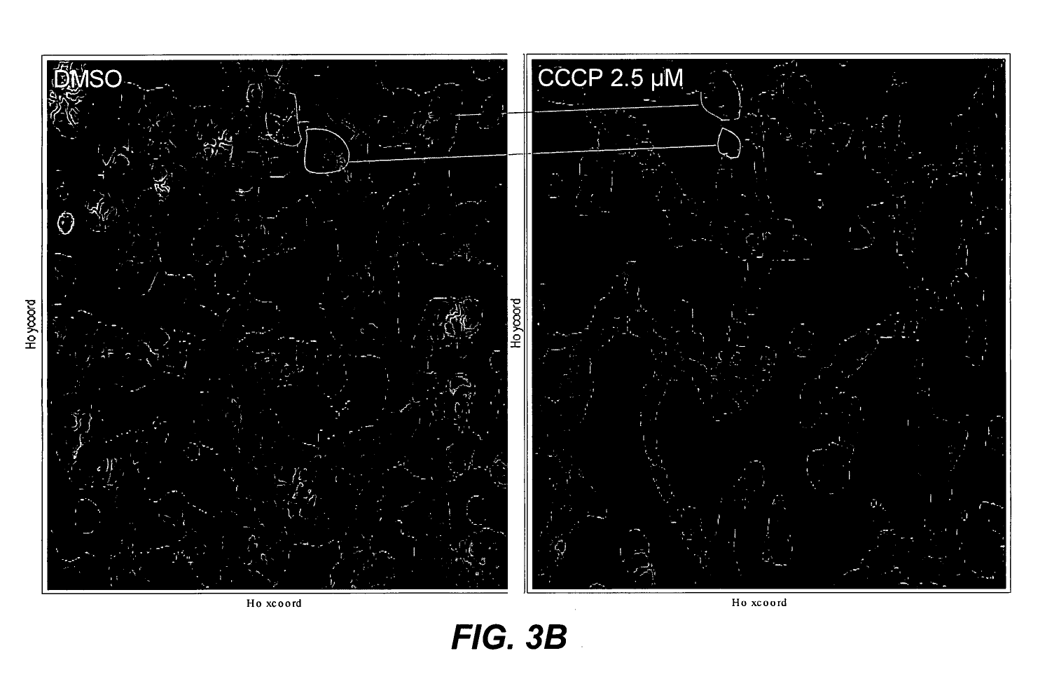

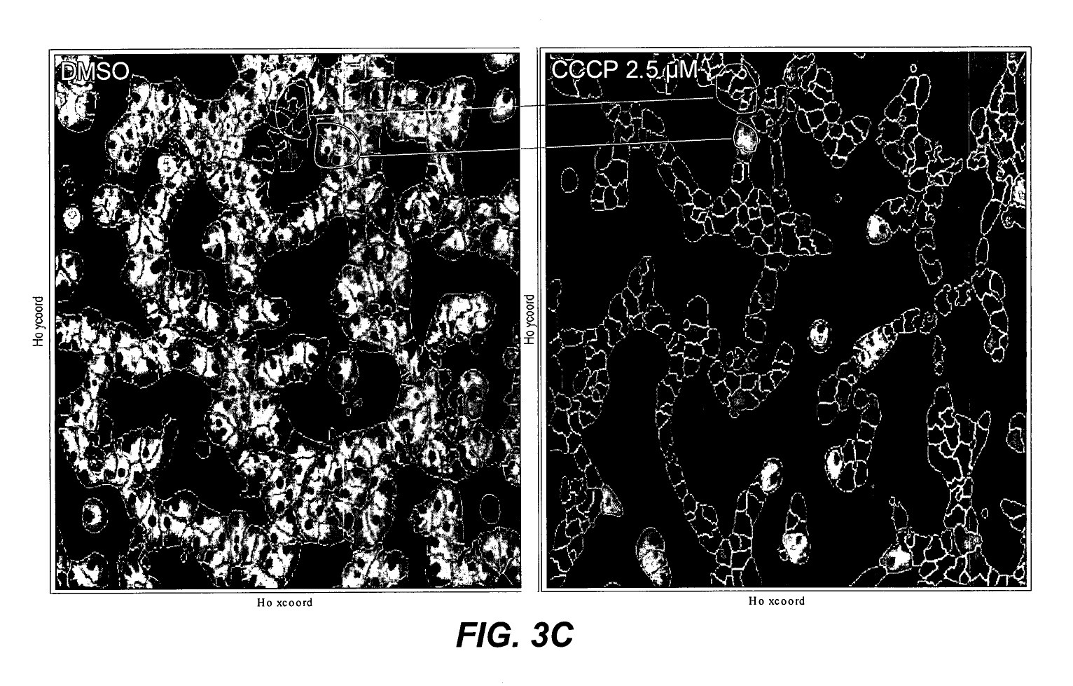

An exemplary two-step segmentation process is illustrated in FIGS. 3A and 3B. FIG. 3A presents the result of the first step. As shown there, two images (the left one for a control population treated with DMSO and the right one for a test population treated with 2.5 μM CCCP) show nuclei circled in the interiors of individual cells. Cellular DNA was stained with Hoechst 33341, which emits fluorescence at a wavelength selectively collected in the FIG. 3A image to permit identification of the individual nuclei. Each such nucleus is presumed to belong to a separate cell.

FIG. 3B presents the results of the second step of the cell segmentation procedure. As shown, the cell populations of FIG. 3A are again presented, but this time at the Alexa 647 channel (i.e., the bright regions in the image locate the source of radiation emitted at the wavelength of Alexa 647). Because this stain shows the location of cellular proteins, the segmentation procedure can locate a cell boundary for each nucleus identified in FIG. 3A. The cell boundaries so identified are circled within the images. Each cell boundary defines a collection of pixels that are deemed to belong to a particular cell. For image processing those pixels are used extracting information about the particular cell in question.

In some embodiments, the segmentation procedure may also identify boundaries of cellular components, e.g. the nucleus and the cytoplasm. Methods for identifying these boundaries from information obtained from images are described in U.S. Pat. No. 6,876,760, titled CLASSIFYING CELLS BASED ON INFORMATION CONTAINED IN CELL IMAGES and U.S. Patent Publication No., 2002-0141631-A1, titled IMAGE ANALYSIS OF THE GOLGI COMPLEX which are hereby incorporated by reference. For example, the boundaries of a nucleus may be identified by applying a gradient and/or threshold technique to DNA signal in an image. The region occupied by a cell's cytoplasm may be identified by removing the region occupied by a nucleus from the total region occupied by the cell.

After the boundaries of each cell have been identified, the appropriate live-dead indicator feature can be extracted on a cell-by-cell basis. See block 209. As indicated above, the intensity of a marker for tubulin (an indicator of local tubulin concentration within the cell) can be identified for each pixel in a given cell. As well, the intensity of other markers such as non-specific protein markers and/or nuclear markers can be identified if appropriate for the analysis routine. Each cell will be characterized on the basis of its level of tubulin or/and other cellular component, whether based on an average value over all pixels in the cell, a maximum or minimum value within the cell, or some other indicator of tubulin quantity. In some embodiments, the mean tubulin marker intensity is calculated over the pixels in a cell and the resulting value is employed as the live-dead indicator feature.

Additional images are presented in FIG. 3C, highlighting the marker DM1-α for tubulin. FIG. 3C shows the same cell populations as in FIGS. 3A and 3B, but at the channel for the wavelength emitted by DM1-α. As indicated previously, the left panel shows a control cell population treated with DMSO and the right panel shows a test cell population treated with 2.5 μM CCCP. Note that live and dead cells can be usually distinguished by visual inspection. Those showing brighter (grey-white) internal regions will be deemed to be live by the methods according to certain embodiments, while those without significant brightness (indicating low levels of tubulin) will be deemed to be dead. While this distinction can be made visually, typical implementations accomplish this automatically, using only computational processing of the data representing the image. In the FIG. 3C images, there is, as expected, a far higher percentage of dead cells in the CCCP treated population than in the control population.

After the data for the cytoskeletal component (or other live-dead indicator) has been produced on a per cell basis, that data is organized or made available in a form that can be used to generate a model for distinguishing live and dead cells. See block 211 of FIG. 2. In a specific example, processing logic provides the live-dead indicator in the form of a histogram showing the number of cells (from the control and test populations) having particular levels of live-dead indicator feature or functions of these levels. In other words, one axis presents various levels of the live-dead indicator feature (or values derived from these levels) and the other axis presents numbers of cells. In some embodiments described herein, the indicator parameter of interest is mean tubulin intensity for a given cell. That is, for any given cell, the tubulin intensity is detected for each and every pixel within the boundary defined for that cell. The mean of the pixel intensities for each cell is then obtained and used as a data point for constructing the histogram. Each cell has its own value of mean tubulin intensity. Cells with higher values of mean tubulin intensity are deemed to be live.

As indicated above, other measures of tubulin may be employed in certain embodiments. For example, in some embodiments, the maximum tubulin intensity found in a cell serves as the live-dead indicator for the cell. In other embodiments, the total tubulin intensity within a cell serves as the indicator. In some embodiments, a function of both a nuclear component (e.g., DNA or histone) and a protein component serves as the indicator. That is, the evaluated function value serves as the indicator.

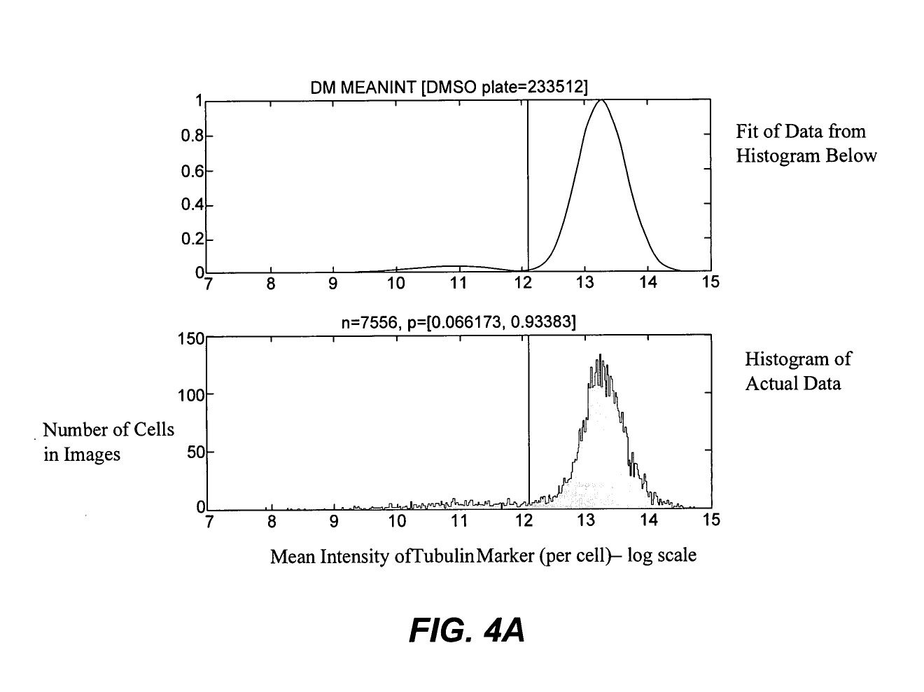

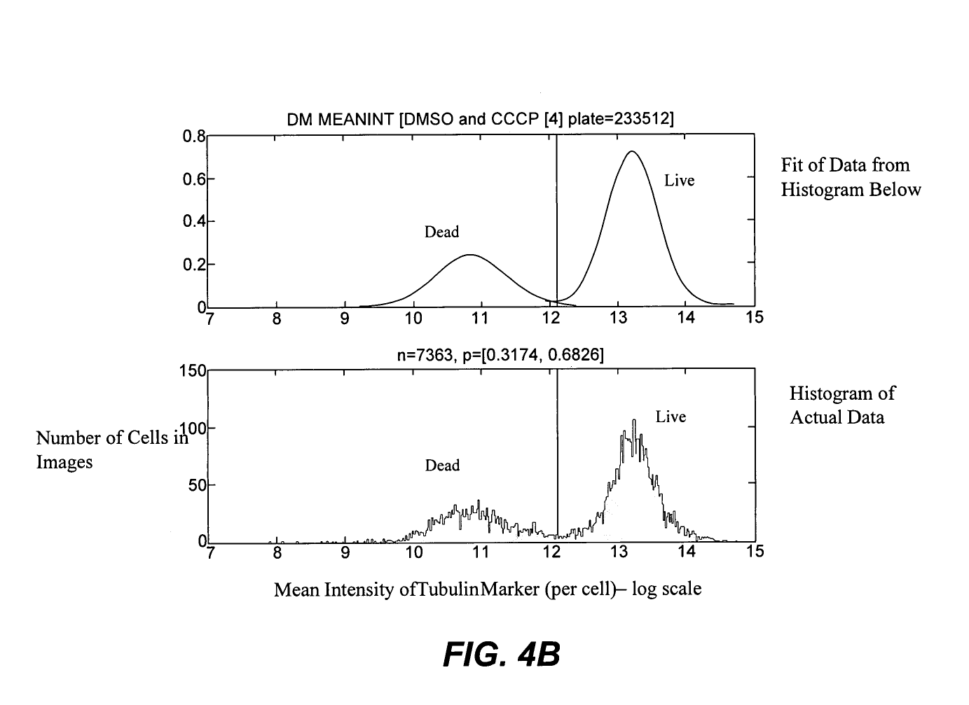

FIGS. 4A and 4B show histograms of mean tubulin marker intensity taken on a per cell basis. The horizontal axis shows the level of mean tubulin marker intensity, with increasingly higher values moving left to right. The vertical axis shows the number of cells found to have particular levels of the mean tubulin marker intensity.

The histogram of FIG. 4A was produced using only control cells treated with DMSO. Thus, in this histogram, most of the data is found in a single peak associated with live cells. In other words, most of the data is found in the right side of the histogram (between the arbitrary values of 12 and 15 on a log scale). However, there is a smaller and wider distribution found to the left of mean intensity value 12. The data in this region of the histogram represents dead cells. As shown in the figure, the raw data is presented in a lower histogram and the “fitted” model is shown in an upper graph. Because the data in the live and dead regions of the histogram is assumed to distribute into two Gaussian distributions, the model produces two Gaussians.

When CCCP treated cells are included together with the control cells, two separate peaks are seen more clearly, each associated with a separate range of mean tubulin marker intensity values. This is illustrated in FIG. 4B where the data in the histogram was taken from test cells as well as control cells. The test cells were treated with various concentrations of CCCP, ranging from 0.625 μM CCCP to 5 μM CCCP for 24 hours. As shown, a relatively large fraction of the cells have a mean tubulin marker intensity well below that associated with the control cells. In other words, the histogram peak associated with dead cells is much more pronounced in FIG. 4B than in FIG. 4A.

As indicated, the models produced using the method depicted here can classify cells as live or dead based on their mean tubulin marker intensity. A confidence can be ascribed to the classification based upon how close the measured intensity value comes to one of the means in the model. Because the model is essentially a “mixture” of two distributions it is referred to as a “mixture model.”

In typical embodiments, the mixture model takes the form of a heterogeneous mixture of Gaussian distributions (e.g., the two Gaussian distributions from the histogram shown in FIG. 4B). Each of these Gaussian distributions may be unambiguously described by the location of its mean and the size of a standard deviation. The models are deemed “heterogeneous” when the two Gaussian distributions are not constrained to have the same values of standard deviation, which is typically the case with models described. As indicated, the mixture model assumes that the data of the training set falls into two distinct Gaussian distributions, one for live cells and the other for dead cells.

Returning to FIG. 2, a mixture model is developed using the training data and one or more a priori constraints. See block 213. In certain embodiments, this involves fitting the indicator data, which is provided in an appropriate format. In addition, constraints on the mixture model (e.g., the number of peaks and the separation of the means of those peaks) are provided. Such constraints are dictated by the underlying biological phenomenon being investigated or deduced empirically. In most instances, a model for distinguishing live and dead cells will be constrained to have two Gaussian distributions, one for live cells and another for dead cells. See the upper panels in FIGS. 4A and 4B. The fact that the model contains two separate Gaussian distributions is an a priori constraint employed to ensure that the resulting model assumes the proper form.

In addition to providing the training data and any necessary constraints, the process may require initial guesses for the various parameters defining the mixture model. Examples of the parameters in question include values of the mean and standard deviation for each Gaussian in the mixture model and additionally the proportions of live and dead cells in the training set. Thus, in one example, the following information is provided with the training set: a number of separate Gaussian distributions (as indicated, two will usually be sufficient), an initial guess for the mean of each Gaussian distribution, an initial guess for the standard deviation of each Gaussian distribution, and an initial guess for the proportion of cells in the training set that are live and the proportion that are dead.

Various types of algorithms may be employed to identify the model parameters using data from the training set. Maximum likelihood estimation is most commonly used approach. The Expectation Maximization (EM) algorithm for maximal likelihood estimation is one suitable numeric likelihood maximization technique. Other maximization techniques may be employed as well. In addition other estimation techniques can be used, such as classical constrained maximum likelihood, MiniMax estimation, and Baysian modelling with estimation using Gibbs sampling. In particular, if distributions other then Gaussian are modelled, an algorithm other than EM may be better suited. Regardless of the particular model generation algorithm employed, the resulting model discriminates between live and dead cells using only mean tubulin intensity (or whatever other particular parameters are identified as the best indicator for distinguishing live cells from dead cells). The model takes the form of two Gaussian distributions, each characterized by the position of a mean and the value of a standard deviation.

In some embodiments, as indicated, the fitting procedure assumes that the mathematical form of the model will be a mixture of Gaussians, and based on this it finds a mean and a standard deviation for each Gaussian. To do this, the procedure employs the multiple constraints (e.g., the number of peaks, the separation of these peaks, etc.). The technique converges after a few iterations of refining the guesses of the means and standard deviations.

At convergence, the maximum likely estimation provides values for the individual means, the individual standard deviations, and the proportions of the live and dead cells in the training set that best fits the data.

As explained, an EM algorithm can be used to find maximum likelihood estimators and hence the most likely values of the means and standard deviations for the distributions in the model. See McLachlan, Geoffrey J., and T. Krishnan (1997), The EM algorithm and extensions, John Wiley and Sons. See also See F. Delaert (2002), The Expectation Maximization Algorithm, College of Computing, Georgia Institute of Technology, Technical Report number GIT-GVU-02-20, both of which are incorporated herein by reference for all purposes.

While tubulin is one suitable live-dead indicator feature, it is not the only feature that can distinguish live cells from dead cells. Note, however, that some other features may require special treatment of living cells. In some embodiments, the living cells are treated with a marker or other agent unrelated to the stimulus under investigation. Living cells are often sensitive to these treatments. Hence, use of indicators working on live cells often requires special handling of the cells and limits the choice of markers to those that do not significantly interfere with normal cell functioning or cellular morphologies to be analyzed. Ideally, as with DM1-α for tubulin, the marker employed for distinguishing live cells from dead cells can be applied to cells immediately before imaging, after they have been fixed. Such markers need not be applied to live cells and thus require no special treatment before cells are fixed, marked, and then imaged.

Of course, the methods described are not limited to such markers. For example, certain embodiments employ a fusion protein of a cell component of interest and a fluorescent protein (e.g., a fusion protein of tubulin and green fluorescent protein or similarly functioning proteins).

In some embodiments, the indicator parameter will have a separate relevance, apart from distinguishing live cells from dead cells. For example, the parameter can indicate an interesting phenotypic characteristic that helps characterize a mechanism of action, a level of toxicity, or other feature under study in conjunction with the live versus dead discrimination. In some embodiments, the indicator parameter will also be used in cell segmentation—e.g., the indicator parameter is a measure of DNA and/or all protein within the cell.

For some applications, tubulin levels meet all the above criteria. A marker such as DM1-α can be applied after the cells are fixed and ready for imaging. It need not be applied while the cells are alive. Further, tubulin and other cytoskeletal components often present interesting morphologies or manifestations of mechanism of action that indicate underlying cellular conditions. Tubulin markers, for example, show the morphology of mitotic spindles and can therefore be used to characterize a cell's mitotic state in some applications—in addition to distinguishing live cells from dead cells. In some embodiments, DNA and protein levels and distributions serve as both indicator parameters and segmentation features.

Models for discriminating live cells from dead cells are used to identify sub-populations of live and dead cells. While such models may be produced in accordance with the methodology described above, this need not be the case. The exact source and development of the model is not critical.

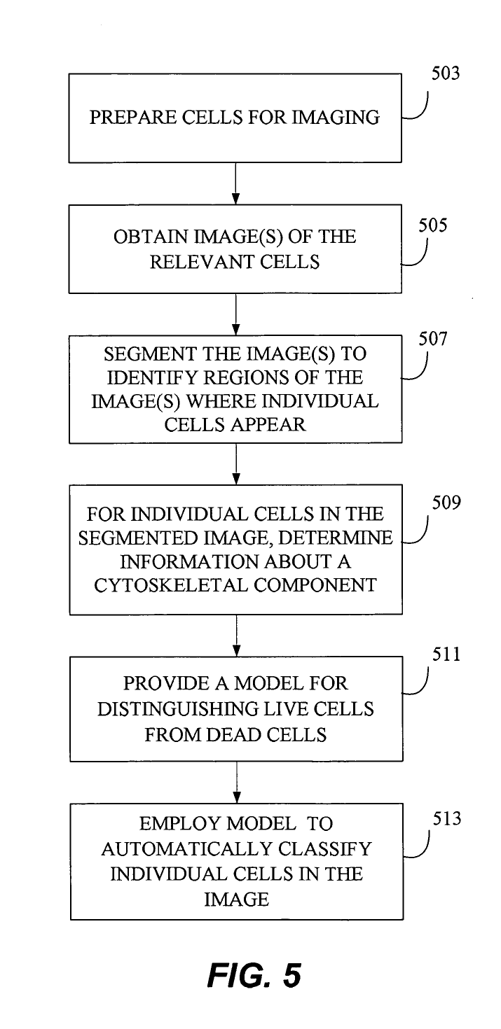

FIG. 5 is a flowchart presenting a typical process for using a model to distinguish live cells from dead cells. In the depicted embodiment, the first four operations of the flowchart shown in FIG. 5 correspond to the first four operations presented in FIG. 2. In FIG. 5, these operations are (1) preparing cells for imaging, (2) obtaining images of the relevant cells and extracting the required features for performing the assay, (3) segmenting the images, including defining boundaries of individual cells, and (4) determining the mean tubulin intensity on a cell-by-cell basis. See blocks 503, 505, 507, and 509. In certain embodiments, the mean tubulin intensity specified in (4) is replaced or supplemented with total intensity of a DNA marker, area occupied by DNA, DNA distribution (e.g., standard deviation of DNA values within a cell), mean intensity of DNA marker within the nucleus, total intensity of a protein marker within the cell, distribution of protein within the cell, and functions of one or more of these.

In FIG. 5, block 511, the process provides a model for distinguishing live cells from dead cells; e.g., a model prepared as described in the context of FIG. 2. Many different types of models can be used, some of which are generated to be widely applicable to different cell types and different assays, and others of which are specific to a very narrow range of samples. In certain embodiments, the model is generated from positive and negative controls known to impact cell populations in different ways, one of which is an effective cell-killing agent.

In certain embodiments, a separate model is generated for each specific condition or assay under consideration. In certain embodiments, a new model is generated for each separate study, involving each separate plate or group of plates. For example, for a given plate the indicator is measured for all cells in all wells. These are then analyzed empirically to identify two distributions, one for live cells and the other for dead cells. The two distributions serve as the model for classifying the cells in the study. In this embodiment, the model is essentially generated on the fly, for each plate or group of plates under consideration and applied to all wells on the plate (i.e., the wells that were employed to generate the model).

Returning to FIG. 5, after the relevant model has been provided or selected, it is applied to the cells. Specifically, the model is employed to automatically classify individual cells in the image on a cell-by-cell basis. See block 513. If a mixture model is employed, application of that model simply involves identifying the mean tubulin marker intensity of a given cell (or other live-dead discriminating feature or function) and determining whether that mean intensity level falls within the Gaussian distribution for live cells or the Gaussian distribution for dead cells. Depending on how close a cell's mean tubulin marker intensity level comes to one or the other of the Gaussian distribution means in the mixture model (and within the standard deviations of those Gaussian distributions), the model may also be able to ascribe some confidence to its classification of the cell in question.

Much of the description in this application refers to cytoskeletal components, such as tubulin, as examples of indicators for determining whether a cell is alive or dead. However, as indicated, other cellular components whose presence or levels within a cell also correspond to live and dead cells may be used. In certain embodiments, models based on DNA and/or protein content of the cell are provided.

Cell death is marked by biological changes to cellular components. These changes may include changes to indicator features such as presence, quantity, distribution, morphology and texture of a particular cellular component in the cell or a region of the cell. Some embodiments discussed above use the presence or quantity of the cytoskeletal protein tubulin as detected using, e.g., the DM1α marker, with death causing a reduced level of tubulin in the cell. In addition to tubulin and other cytoskeletal proteins, cell death may also be marked by changes involving DNA and total protein content within a cell. Specific examples of changes to the presence, quantity, distribution, morphology and texture of these components that cells may undergo when they die include the following:

1) The total amount of DNA within a cell may decrease.

2) The DNA in the nucleus becomes more condensed, i.e., the DNA occupies a smaller area.

3) The distribution of DNA within the nucleus becomes less uniform. DNA distribution in live cells is typically flat or uniform in the nucleus. At death, the DNA may become more uneven. This uneven distribution may take several forms. Often the DNA will appear fragmented, punctate, i.e. with small holes interrupting the flat distribution, or donut-shaped or toric.

4) The amount of total protein within the cell decreases.

5) The distribution of protein between the nucleus and the cytoplasm changes. Dead cells have relatively more proteins in the nucleus than the cytoplasm. While most cells undergo at least some of the above biological changes when they die, whether and how a cell undergoes a particular change can depend upon the pathway of death (apoptosis, necrosis, etc.).

In certain embodiments, DNA and protein-based models may be generated based on some or all of the above indicators of whether a cell or a population of cells is alive or dead. Various features extracted from images on a per cell basis relate to one or more of the above biological changes associated with cell death. These features can be used alone or in combination to provide a model for cell death. In some embodiments, the features are represented as variables in expressions (sometimes referred to herein as indicator expressions), which provide an estimate of whether a cell shown in an image was alive or dead. Thus, such expressions serve as models for predicting whether a cell was alive or dead.

According to various embodiments, the models may be linear or non-linear combinations of variables representing one or more of these features. The variables or features representing these changes may be obtained from information about the DNA and protein present in images of the cells. Similar to the tubulin-based models, relevant features may be extracted from images by detecting pixel intensity in specific channels, which intensity represents DNA or protein content associated with appropriate markers. Examples of such features that may be used to represent each of the biological changes are described below.

The amount of DNA in a cell and how it is distributed are indicators of cell death. Nuclei may become smaller in dead cells. This may be represented by the median or mean intensity of the DNA marker signal across the pixels within a nuclei's boundaries.

The area that the nucleus occupies within a cell is an indicator of cell death because the DNA condenses when a cell dies. This area may be represented by the area within a nucleus occupied by pixels having a DNA signal greater than a threshold value. This requires identifying the boundary of the nucleus. In certain embodiments, the nucleus boundary may be identified by calculating the gradient of pixel intensity and thresholding. This is typically done as part of the segmentation procedure described above.

The distribution of the DNA within the cell or nucleus is an indicator of cell death because the DNA sometimes fragments or otherwise redistributes when a cell dies. This condition may be represented by the standard deviation of the DNA pixel values within cell boundaries or nucleus. While DNA distribution may be represented by the standard deviation of the DNA pixel values, it could also be represented by texture features (described below) or by parameters related to higher order moments alone or in combination with the standard deviation. For example, kurtosis or the fourth moment of the DNA pixel values is a measure of peakedness and may be used to represent DNA distribution.

The total amount of protein in a cell is an indicator of cell death because it decreases when a cell dies. The total protein may be represented by the total intensity of the protein marker signal over all pixels within cell boundaries.

The distribution of protein between the nucleus and the cytoplasm is an indicator of cell death because the protein redistributes when a cell dies Dead cells have decreased protein content in cytoplasms and no change or increase in nucleus. The protein distribution may be represented by the intensity of the protein marker signal within the nucleus boundaries relative to that within the cytoplasm boundaries. (In certain embodiments, identifying the cell boundaries involves using Alexa 647 data and may be done as part of the segmentation procedure described above.) Protein distribution may also be represented using one or more moments of the protein pixel signal intensity (e.g., variance or standard deviation, skewness, kurtosis), either alone or in combination with the relative intensity.

As with DNA distribution, texture features may also be used to represent protein distribution. Texture features may characterize cell components within an area of an image, typically the area identified as a cell or a nucleus. Examples of ways to classify texture include directional v. non-directional, smooth v. rough, coarse v. fine and regular v. irregular. Texture features may, for example, be used to distinguish a smooth or uniform region of DNA from a punctate region. Statistical methods that may be used to generate texture features from an image include Co-occurrence Matrix, Autocorrelation, Power Spectrum (Frequency Domain) and Grey Level Run Length. Geometric methods that may be used to generate texture features include texture primitives (tokens) extraction. These include Edge Detection or Adaptive Region Extraction, Voronoi Tessellation and Structure Methods. Model-based methods include Markov Random Fields (in which the intensity of each pixel depends only on the intensities of the neighboring pixels), Fractal Methods and Multi-resolution Auto-regression (a linear regression of a pixel intensity given the intensities in its neighbourhood). Signal processing methods include Spatial Domain Filters, Frequency Domain Filters, and Gabor and Wavelet Models.

As mentioned above, a particular cell may undergo only some of the changes describe above. Using a combination of DNA and protein features may be desirable to capture a range of cell death pathways. Thus, while embodiments of the tubulin-based models described above are based on a single feature or indicator (e.g., mean tubulin intensity), the DNA and protein-based models are typically based on an indicator expression which is a combination of features. For example, the indicator expression may take the form: P=c₁F₁+c₂F₂+ . . . cₙFₙ+k, where cₙ is a coefficient, Fₙ a feature, k a constant, and P is an indicator of cell death. The combination may also be non-linear. P may represent a probability or related property of a cell being dead (or alive). In some embodiments, P simply represents a binary decision—e.g., if P is less than or equal to some value, the cell is said to be dead and if P is greater than that value, the cell is said to be alive. In some embodiments, P may be a surrogate for another indicator of a cell being dead (e.g., P may represent a mean tubulin intensity).

In addition to the features listed above (total intensity of DNA pixels within cell or nucleus boundaries, DNA area, mean intensity of DNA, standard deviation (or variance or higher order moments) of DNA pixel intensity, total intensity of protein pixels, relative amount of protein pixel intensity in the nucleus vs. the cytoplasm, standard deviation (or variance or higher order moments) of protein pixel intensity, and DNA or protein texture features), other features may be used. These include, for example, total, mean, or higher order moments of DNA or protein intensity in the nuclei, cell or cytoplasm, morphological features including cell and nucleus area, diameter and elliptical axes ratios. Such features when used alone or in combination with other features provide an indication of whether the cell was alive or dead.

As indicated above, in certain embodiments, DNA and/or protein markers provide signals to be captured in the image (e.g., a fluorescent emission). DNA content of a cell or region of a cell may be measured using markers such as DAPI or Hoechst 33341 stains. One of skill in the art will understand that other markers that co-located with protein or DNA may be used as well. The overall protein content of a cell or region of a cell may be measured using the Alexa 647 succinimidyl ester (Alexa 647) marker, or another marker that co-locates with all or most cellular protein.

DNA and protein-based models may be desirable in various situations. For example, the fluorescently labeled antibodies to tubulin (e.g., DM1-α, YL1-2, and 3A2 antibodies) that may be used as tubulin markers may not be appropriate for certain assays.

These and other antibody markers typically require a detergent to penetrate the cell membrane. Introduction of the detergent (after fixing the cells) disrupts the cell membrane. Thus, certain applications, e.g., imaging membrane lipids, must be conducted without use of detergent-based markers. The Hoechst and Alexa 647 markers described above can be used without detergent, and therefore may be used in conjunction with lipid assays and other assays where the use of a detergent would not be acceptable.

In addition, the DNA and protein-based models are useful for applications in which there is a limited number of channels available for imaging. As described above, DNA and protein channels may be used to segment the cells; using the imaging data obtained from these channels for a live/dead assay obviates the need to dedicate an additional channel to a marker used solely for the live/dead assay. Thus, for example, if four channels are available, two channels may be used for other markers necessary for other assays.

Models employing DNA and protein feature for distinguishing live and dead cells may take any form described above. They may be mixture models, decision trees, linear expressions, non-linear expressions, etc. Assuming that such a model takes the form of a mixture model, it may be used essentially as described above in the discussion of FIG. 5. Of course the use of a tubulin feature (or feature of other cytoskeletal component) is replaced with use of DNA and total cellular protein features. Further, the use of these features is provided in combination, such that these features serve as values for independent variables in an expression that is evaluated to give a result that is applied to the mixture model.

One method for generating DNA and protein-based models involves the following steps: (1) preparing cells for imaging, (2) obtaining images of the relevant cells and extracting the required cellular features for performing the analysis, (3) segmenting the images, including defining boundaries of individual cells and regions of the cells (e.g., nuclei and cytoplasm) and extracting relevant DNA and protein features on a per cell basis, (4) evaluating an indicator expression containing the relevant DNA and protein features to obtain an indicator value for each cell in the segmented images, (5) presenting the indicator value for each cell as training data, and (6) developing a mixture model using the training data and a priori constraints on the model.

Many of these steps are the same or similar as those employed to generate the tubulin-based models described above, the main difference being that an indicator expression based on DNA and protein information is used to generate the model, instead of a simple feature value; for example, mean tubulin intensity. Specifically, preparing and imaging the cells, segmenting the images and developing the mixture model using the training set data may be performed generally as described above (making appropriate changes for using DNA and total protein content information instead of tubulin information).

As with the tubulin-based model, preparing the cells for imaging involves treating a control population of cells with a control compound (e.g., DMSO) and test population of cells treated with a compound or stimulus known to kill a significant percentage of cells (e.g., CCCP). In certain embodiments, the control and test populations are provided on designated wells of a particular plate. The model then may be generated for all wells on that plate (or group of plates).

Upon completion of treatment, cells are fixed and stained with appropriate markers. Unlike the tubulin-based model, the Hoechst and Alexa markers (or other appropriate DNA and protein markers) are employed to perform both segmentation and the live/dead assay.

Imaging the cells may also be performed largely as described above with respect to the tubulin-based model. From the cellular images, the process extracts multiple cellular features, at least one of which allows segmentation of the cells and at least one of which provides a measure of the DNA and/or total protein content over the cell. Typically, both DNA and total protein content are extracted. In some cases a morphological indicator of interest is also taken with the image (e.g., the trans-Golgi network marker shown in FIG. 1).

One method of segmentation is described above with respect to FIGS. 3A and 3B in which Hoechst and Alexa 647 markers are used to identify cell boundaries. In addition to identifying cell boundaries, in certain embodiments, the segmentation process used to generate DNA and protein-based models also identifies nucleus and/or cytoplasm boundaries (e.g., to find the relative protein distribution in the nucleus and the cytoplasm).

DNA and total protein signal intensity data are then also used to calculate the indicator expression. Once the indicator expression is calculated for each cell, a mixture model may then be generated as discussed above with reference to FIGS. 4A-C. As explained, in DNA and protein models, the histograms and Gaussian distributions represent an indicator value or result calculated from the indicator expression, instead of the simple mean or total tubulin intensity described above.

As indicated above, the indicator expression may be a combination of DNA and total protein-related features (mean intensity, standard deviation of intensity, etc.). An example of the indicator expression is given above: P=c₁F₁+c₂F₂+ . . . cₙFₙ+k, where cₙ is a coefficient, Fₙ a feature, k a constant, and P is an indicator of cell death. P may represent a probability or related property of a cell being dead (or alive). Identifying which features to use in the expression, the form of the expression (linear, non-linear, values of coefficients and constants) may be done by various techniques including statistical techniques such as model building (or variable selection) based on bootstrap samples. Features may be also be identified based on biological observation and knowledge. The expression should provide an indication of whether the cell is alive or dead. Two examples, one for detergent-based assays and one for non-detergent based models are given below.

### EXAMPLE I

### Detergent-Based Model

A model was constructed for detergent-treated hepatocyte cells. The model was validated using mean tubulin intensity as measured by DM1-α. An indicator expression of the form P=c₁F₁+c₂F₂+ . . . cₙFₙ+k was derived, with P being the mean tubulin intensity (used as a live/dead surrogate) and the feature values being mean DNA intensity (as measured by the Hoechst marker), mean protein intensity (as measured by Alexa) and standard deviation of DNA intensity. In some embodiments, the general form of the expression is as follows:
DM.meanint=f(HO.meanint, A647.meanint, HO.stdint),
with DM.meanint being mean tubulin intensity, HO.meanint being mean Hoechst signal intensity (Hoechst 33341 marker for DNA), A647.meanint being mean Alexa 647 signal intensity and HO.stddev being the standard deviation of the Hoechst intensity. In some embodiments, the derived expression has the following form:
log₂(DM.meanint)≈0.1305 log₂(HO.meanint)+1.0622(A647.meanint)−0.0002(HO.stdint)−3.2793

The features and coefficients of the indicator expression were obtained by taking a large number of Hoechst and Alexa-related features (approximately 30) and performing variable selection to find the variables or features that were most predictive of the live/dead outcome. Variable selection may be performed by any known method. In this case, DM1α intensity data was collected and used as a surrogate measure of the live/dead outcome (based on the tubulin model previously generated and described above). As noted Hoechst 33341 mean intensity, Alexa mean intensity, and standard deviation of the Hoechst intensity were selected. The coefficients were determined using a regression method on a training set. Coefficient values were estimated for a large number of samples and the average of the estimated values to determine the coefficients in the expression.

To generate the final form of the model, the indicator expression was evaluated on a per-cell basis, and a mixture model was generated in the same manner as described above with reference to FIGS. 4A and 4B. Briefly, for any given cell, the DNA (as marked by the Hoechst dye) and total protein intensity (as marked by the Alexa dye) was detected for each and every pixel within the boundary defined for that cell. The mean of all protein pixel intensities, the mean of all DNA pixel intensities, and the standard deviation of all DNA pixel intensities for each cell was then obtained and used to evaluate the indicator expression—in this case an estimate of the mean tubulin intensity. Each cell had its own value of estimated log₂ (DM.meanint). Cells with higher values of estimated log₂(DM.meanint) were deemed to be live. The values of the indicator expression were used as data points to provide an empirical distribution of the level of the log₂(DM.meanint) in individual cells, which can be visualized as a histogram of the number of cells in the images versus the log₂(mean tubulin intensity) in an individual cell. Using this empirical distribution, a mixture model of the two Gaussian distributions was then determined. In certain embodiments, an Expectation Maximization (EM) procedure is used to identify a mean and a standard deviation for each of the two Gaussian distributions.

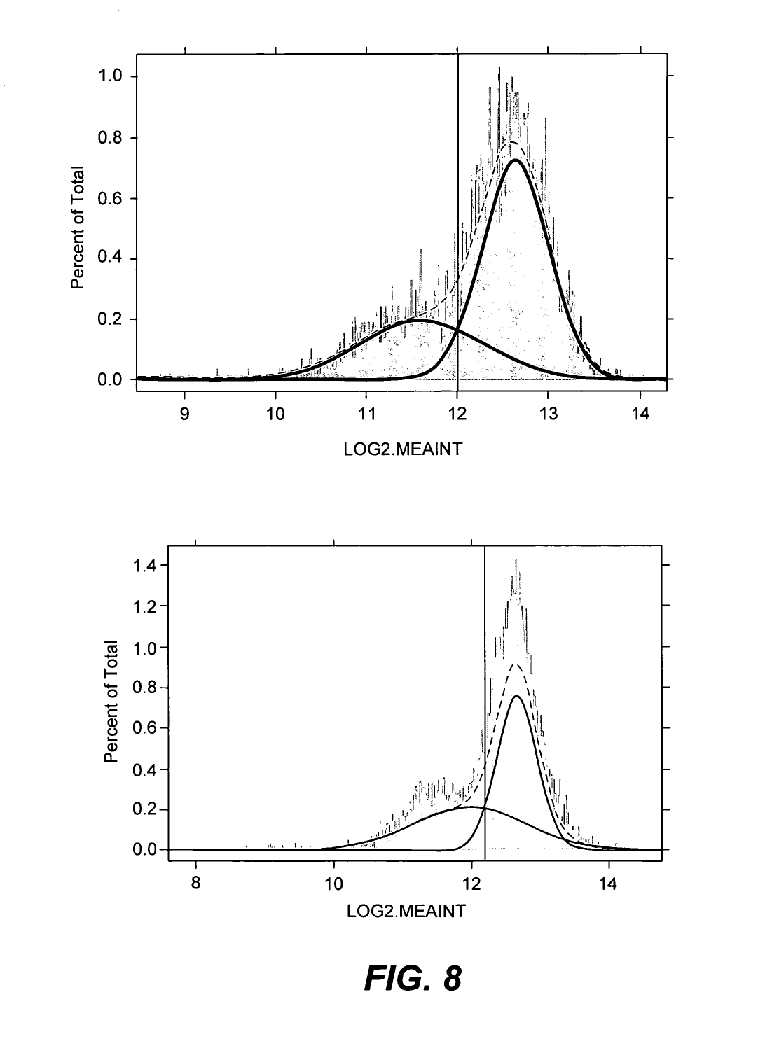

FIG. 8 shows histograms of log₂ (DM.meanint) for two sets of control and test cells. The curves were generated from data obtained from a different assay. Each assay obtained data from total protein and DNA markers in addition to other markers that differ from assay to assay. The Gaussian curves generated from the EM procedure are also shown on the curves. The vertical line shows where the curves intersect; this value may be used to determine whether a cell is live or dead when the model is applied as described above with reference to FIG. 5. There is clear separation of the modes for all of the distributions. The difference is the histograms is expected and is due to differences in each of the assays; however regardless of the particular assay run the model produces a clear separation of the live and dead cells.

### EXAMPLE II

### Non-Detergent Treated Model

A different model may be required for cells not treated with detergent. As indicated, treating a cell with detergent disrupts the cell membrane, which allows some markers to penetrate the cell but may limit applicability of the assay. Because the markers used in the assay may penetrate the cell differently when the cell membrane is intact, a different indicator expression and model is used for non-detergent treated cells. Alexa 647 in particular does not penetrate the cell interior as well when the cell membrane is intact.

A non-detergent treated model was generated using an indicator expression having the form In (P/(1−P))=c₁F₁+c₂F₂+ . . . cₙFₙ+k. In this case, P is the probability of a cell being dead, and P/(1−P) is an odds ratio, also referred to as the “logit.” The following features were used:
standard deviation of the DNA intensity (as measured by the Hoechst marker) within the nucleus (stdint.HO.ContourTypeDna)
total DNA intensity within the nucleus (totalint.HO.ContourTypeDna)
area of the nucleus as measured by the number of pixels (area.ContourTypeDna)
total protein intensity (as measured by the Alexa marker) within the cell (totalint.A647.ContourTypeCell)
mean protein intensity within the cell (meanint.A6487.CoutourTypeCell)
mean protein intensity within the cytoplasm (meanint.A647.CountourTypeCytoplasm)

The derived expression has the following form:
log (P/(1−P))≈−0.00275(stdint.HO.ContourTypeDna)+0.34672 log₂(totalint.HO.ContourTypeDna)+9.43676 log₂ (area.ContourTypeDna)−0.32484 log₂ (totalint.A647.ContourTypeCell)−5.55039 log₂ (meanint.A6487.ContourTypeCell)+7.66012 log₂ (meanint.A647.ContourTypeCytoplasm)

As with the detergent treated model, the indicator expression was determined by applying variable selection to select from multiple Hoechst and Alexa-related variables. Coefficients were found by fitting the data to the known outcomes. Unlike the detergent treated model, DM1-α could not be used to generate or validate the non-detergent model. Instead DMSO-treated (negative control) cells were assumed alive and CCCP-treated (positive control) cells were assumed dead.

The value of In (P/(1−P)) was calculated for each cell and the EM algorithm was used to estimate a threshold. It should be noted that a model based on the above expression will give a probability a cell being dead or alive (when applied to cells). It is not necessary to use the constant k when using this general approach to generate a mixture model. Similarly, in calculating other indicator expressions (for example, for another cell line), it is not necessary to calculate the constant. Calculating this constant is necessary only if it desired to obtain a probability that the cell is dead, rather than merely classifying the cell as dead or alive based on the threshold determined by the mixture model.

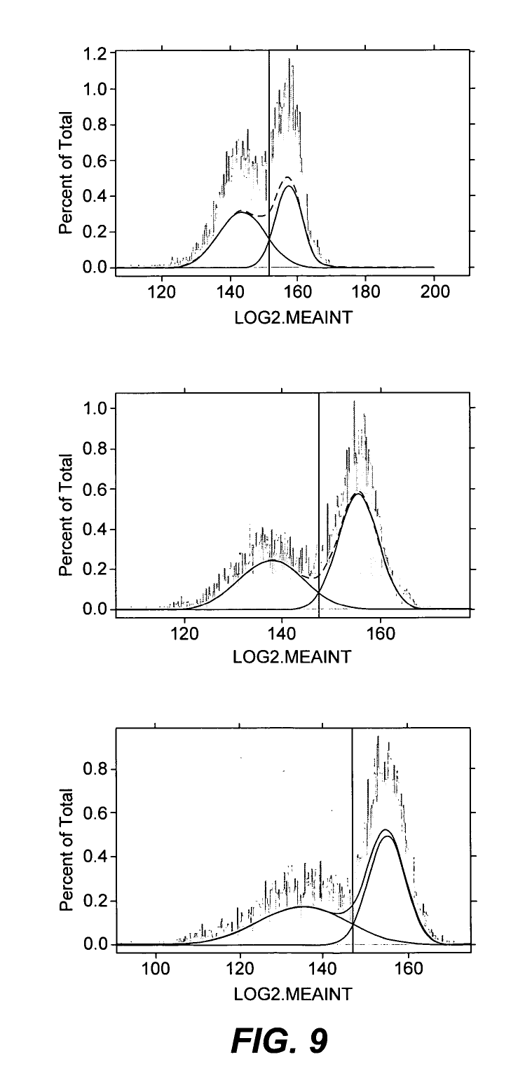

FIG. 9 shows histograms of log (P/(P−1)) for three sets of control and test cells; as in FIG. 8, each histogram corresponds to data generated from a different assay. The Gaussian curves generated from the EM procedure are also shown on the curves. The vertical line shows where the curves intersect; this value is typically used to determine whether a cell is live or dead when the model is applied as described above with reference to FIG. 5.

The indicator expressions such as those given above for the detergent and non-detergent treated models may be used to generate models. Modifications to imaging and staining procedures may result in modifications to the coefficients. Similarly, different types of cells may result in modifications to the coefficients and possibly to the variables or features selected for use in the expressions. Thus, for a particular imaging and staining protocol it may be necessary to generate an indicator expression to be used in generating and applying models using that protocol. Similarly, for a particular cell line, it may also be necessary to generate an indicator expression.

As should be apparent, the methods and other aspects described have many different applications. In certain applications, the percentages or absolute numbers of live and dead cells in samples that have been treated with particular stimuli may be determined. One extension of this basic application produces a “stimulus-response” characterization in which increasing levels of applied stimulus are employed (e.g., increasing concentration of a particular drug under investigation). The proportions of live and dead cells are then observed to change with changing levels of the stimulus. This may indicate the potency of the stimulus, its mechanism of action, etc. See for example, U.S. patent application Ser. No. 09/789,595, filed Feb. 20, 2001, titled CHARACTERIZING BIOLOGICAL STIMULI BY RESPONSE CURVES and U.S. Provisional Patent Application No. 60/509,040, filed Jul. 18, 2003, titled CHARACTERIZING BIOLOGICAL STIMULI BY RESPONSE CURVES, both of which are incorporated herein by reference for all purposes.

In certain applications, the the live versus dead discrimination may be applied to more clearly characterize some morphological change arising from a given stimulus. Such change may be more pronounced in one or the other of live and dead cells. In fact, some morphological effects might affect only live or dead cells (or might affect them in fundamentally different ways). A raw analysis of such effect on an entire population of cells—that includes both live and dead cells—without separately considering the effect on live and dead cells could mask the specific impact of the stimulus on live or dead cells.

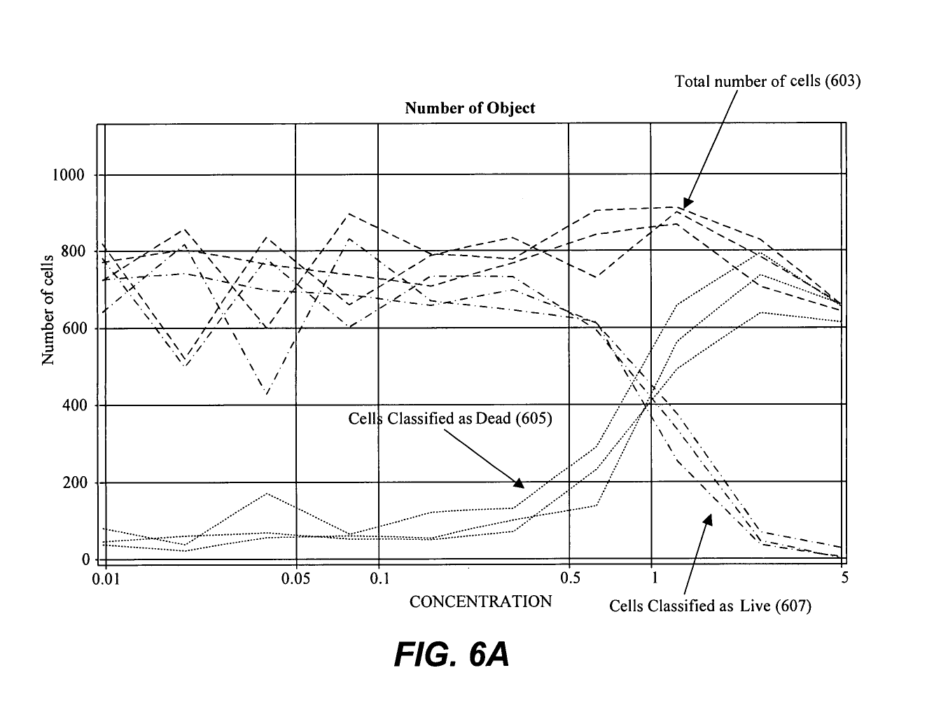

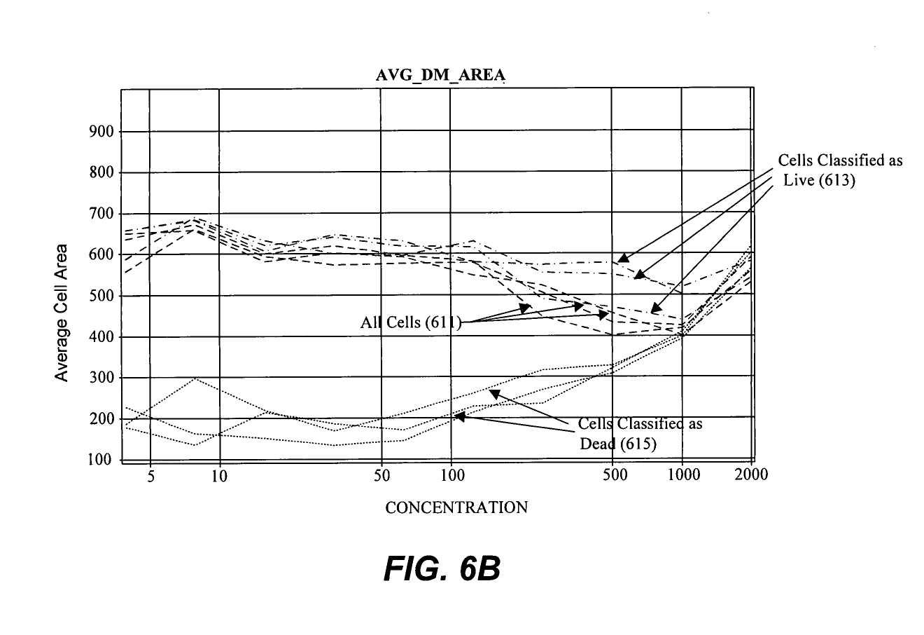

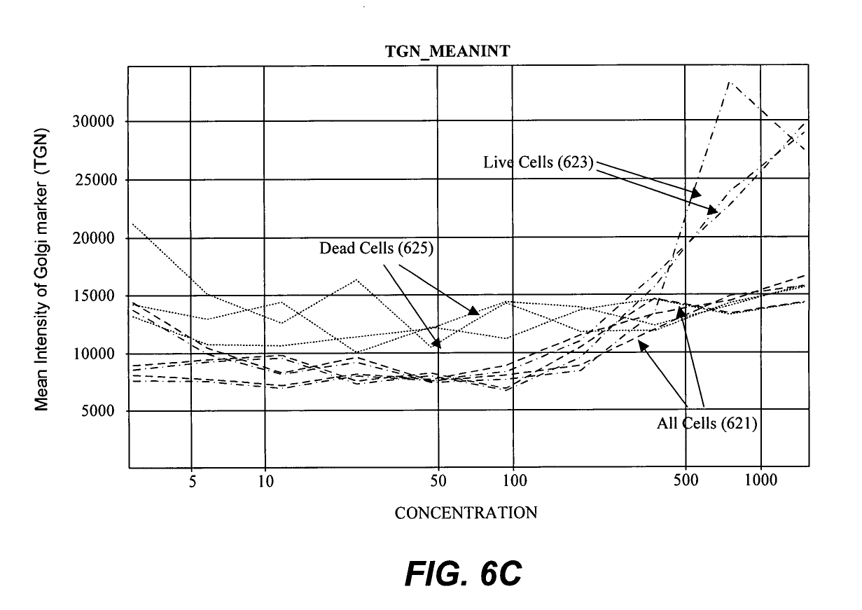

In view of the above, the flowchart of FIG. 5 may be extended to include an additional operation in which the automated image processing extracts a feature (sometimes in addition to the ones required for segmentation and live-dead discrimination) from the cell images on a cell-by-cell basis. The Golgi feature shown in FIG. 1 is one example of such additional feature. In this additional operation, the image analysis algorithm determines how the additional feature is separately manifest in the live and dead cell populations. Examples of cellular/morphological conditions found to be exhibited differently in live and dead cells are presented in FIGS. 6A through 6C.

FIG. 6A shows how CCCP concentration affects the total number of cells in an image, as well as the number of dead cells and the number of live cells. In this plot, the total number of cells (603) is shown to remain approximately constant across three different samples of around 800 cells each. These are identified as a number of objects (determined by the number of DNA spots). Three paths 605 show the number of dead cells, while three paths 607 show the number of live cells. As shown, starting at a normalized concentration of about 0.5 μM CCCP, the number of dead cells begins to increase dramatically, while the number of live cells begins to decrease at roughly the same rate. Note that the live cells were discriminated from dead cells using the mean DM1-α intensity of each cell. Other techniques for discriminating between live and dead cells—such as the DNA-protein models described above—could also be used.

FIG. 6B shows the effect of a different drug (diclofenac) on the total cell area. The area was determined from a pixel count within the boundary determined by segmentation. Of particular interest, this plot shows that the area of the dead cells 615 begins to increase rather dramatically at a particular concentration (the arbitrary concentration of 100 shown in this plot). In contrast, the total area of the live cells 613 gradually decreased beginning at approximately the same concentration. A simple consideration of the average cell area for all cells (including live cells and dead cells) would likely mask the fact that the higher concentrations of the drug cause dead cells to become progressively larger. The effect of the live cells masks, somewhat, the effect on the dead cells. This result would not have been observed without the assay provided, which distinguishes live cells from dead cells. Note that the average area of the live and dead cells together is shown as lines 611 in FIG. 6B. Note also that, as with the plot in FIG. 6A, the live cells were discriminated from dead cells using the mean DM1-α intensity of each cell. Again, other techniques for discriminating between live and dead cells could be employed with equal effect.

Finally, FIG. 6C shows how another drug (tacrine) impacts the mean intensity (on a cell-by-cell basis) of a marker for the TGN (trans-Golgi network). Of interest in this plot, increasing concentrations of tacrine dramatically increase the mean Golgi marker intensity signal in live cells (lines 623), while having a relatively minimal affect on the Golgi intensity signal in dead cells (lines 625). This is another situation in which simply considering the live and dead cells together (the data paths 621) would have masked the separate effect of the drug on a morphological indicator (mean TGN marker intensity) on a separate class of cells (the live cells). As with FIGS. 6A and 6B, the live-dead discrimination in FIG. 6C was made using mean DM1-α intensity (per cell).

Certain embodiments employ processes acting under control of instructions and/or data stored in or transferred through one or more computer systems. Certain embodiments also relate to an apparatus for performing these operations. This apparatus may be specially designed and/or constructed for the required purposes, or it may be a general-purpose computer selectively configured by one or more computer programs and/or data structures stored in or otherwise made available to the computer. The processes presented herein are not inherently related to any particular computer or other apparatus. In particular, various general-purpose machines may be used with programs written in accordance with the teachings herein, or it may be more convenient to construct a more specialized apparatus to perform the required method steps. A particular structure for a variety of these machines is shown and described below.

In addition, certain embodiments relate to computer readable media or computer program products that include program instructions and/or data (including data structures) for performing various computer-implemented operations associated with analyzing images of cells or other biological features, as well as classifying stimuli on the basis of how they impact cell viability or selectively act on subpopulations of cells. Examples of computer-readable media include, but are not limited to, magnetic media such as hard disks, floppy disks, and magnetic tape; optical media such as CD-ROM disks; magneto-optical media; semiconductor memory devices, and hardware devices that are specially configured to store and perform program instructions, such as read-only memory devices (ROM) and random access memory (RAM). The data and program instructions provided herein may also be embodied on a carrier wave or other transport medium (including electronic or optically conductive pathways).

Examples of program instructions include low-level code, such as that produced by a compiler, as well as higher-level code that may be executed by the computer using an interpreter. Further, the program instructions may be machine code, source code and/or any other code that directly or indirectly controls operation of a computing machine. The code may specify input, output, calculations, conditionals, branches, iterative loops, etc.

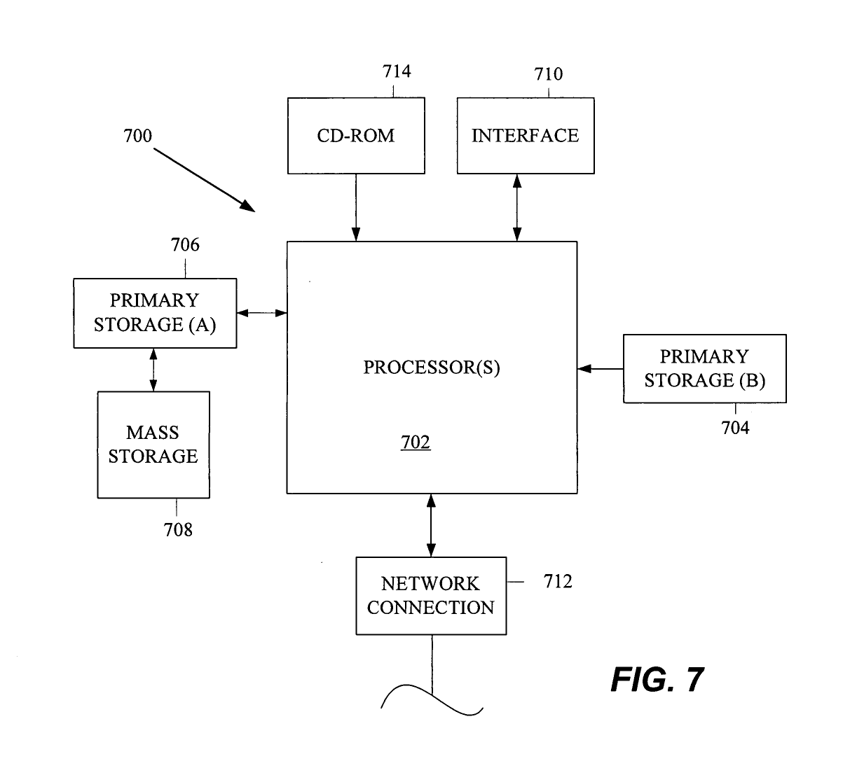

FIG. 7 illustrates, in simple block format, a typical computer system that, when appropriately configured or designed, can serve as a computational apparatus according to certain embodiments. The computer system 700 includes any number of processors 702 (also referred to as central processing units, or CPUs) that are coupled to storage devices including primary storage 706 (typically a random access memory, or RAM), primary storage 704 (typically a read only memory, or ROM). CPU 702 may be of various types including microcontrollers and microprocessors such as programmable devices (e.g., CPLDs and FPGAs) and non-programmable devices such as gate array ASICs or general-purpose microprocessors. In the depicted embodiment, primary storage 704 acts to transfer data and instructions uni-directionally to the CPU and primary storage 706 is used typically to transfer data and instructions in a bi-directional manner. Both of these primary storage devices may include any suitable computer-readable media such as those described above. A mass storage device 708 is also coupled bi-directionally to primary storage 706 and provides additional data storage capacity and may include any of the computer-readable media described above. Mass storage device 708 may be used to store programs, data and the like and is typically a secondary storage medium such as a hard disk. Frequently, such programs, data and the like are temporarily copied to primary memory 706 for execution on CPU 702. It will be appreciated that the information retained within the mass storage device 708, may, in appropriate cases, be incorporated in standard fashion as part of primary storage 704. A specific mass storage device such as a CD-ROM 714 may also pass data uni-directionally to the CPU or primary storage.

CPU 702 is also coupled to an interface 710 that connects to one or more input/output devices such as such as video monitors, track balls, mice, keyboards, microphones, touch-sensitive displays, transducer card readers, magnetic or paper tape readers, tablets, styluses, voice or handwriting recognition peripherals, USB ports, or other well-known input devices such as, of course, other computers. Finally, CPU 702 optionally may be coupled to an external device such as a database or a computer or telecommunications network using an external connection as shown generally at 712. With such a connection, it is contemplated that the CPU might receive information from the network, or might output information to the network in the course of performing the method steps described herein.

In one embodiment, a system such as computer system 700 is used as a biological classification tool that employs gradient determination, thresholding, and/or morphology characterization routines for analyzing image data for biological systems. System 700 may also serve as various other tools associated with biological classification such as an image capture tool. Information and programs, including image files and other data files can be provided via a network connection 712 for downloading by a researcher. Alternatively, such information, programs and files can be provided to the researcher on a storage device.

In a specific embodiment, the computer system 700 is directly coupled to an image acquisition system such as an optical imaging system that captures images of cells or other biological features. Digital images from the image generating system are provided via interface 712 for image analysis by system 700. Alternatively, the images processed by system 700 are provided from an image storage source such as a database or other repository of cell images. Again, the images are provided via interface 712. Once in apparatus 700, a memory device such as primary storage 706 or mass storage 708 buffers or stores, at least temporarily, digital images of the cells. In addition, the memory device may store phenotypic characterizations associated with previously characterized biological conditions. The memory may also store various routines and/or programs for analyzing and presenting the data, including identifying individual cells as well as the boundaries of such cells, characterizing the cells as live or dead, extracting morphological features (e.g., the shape of mitotic spindles), presenting stimulus response paths, etc. Such programs/routines may encode algorithms for characterizing intensity levels at various channels, performing thresholding and watershed analyses, performing statistical analyses, identifying edges, characterizing the shapes of such edges, performing path comparisons (e.g., distance or similarity calculations, as well as clustering and classification operations), principal component analysis, regression analyses, and for graphical rendering of the data and biological characterizations.

Although the above has generally described certain embodiments according to specific processes and apparatus, the subject matter of the description provided has a much broader range of implementation and applicability. Those of ordinary skill in the art will recognize other variations, modifications, and alternatives.

What is claimed is:

1. A method of distinguishing live cells from dead cells in a population of cells, the method comprising:
(a) providing one or more images of at least one cellular component in a population of cells;
(b) automatically analyzing said one or more images to determine, for at least some cells in said population of cells, information about the at least one component; and
(c) automatically using the information about the at least one component to classify said at least some cells as live or dead;
wherein the at least one cellular component is selected from cellular protein and DNA.

2. The method of claim 1, wherein the information about the cellular component comprises information about at least one of the total amount, area or distribution of the cellular component in the cell or a region of the cell.

3. The method of claim 1, wherein the information about the cellular component comprises intensity levels for a marker of the component shown in the image.

4. The method of claim 1, wherein the information about the cellular component comprises at least one of the mean intensity or a moment of the intensity of the marker.

5. The method of claim 1, wherein the information comprises a combination of the mean intensity of a DNA marker within the cell, the standard deviation of the intensity of the DNA marker within the cell, and the mean intensity of a cellular protein marker within the cell.

6. The method of claim 1, wherein the information comprises a combination of the standard deviation of the intensity of a DNA marker within the cell, the total intensity of the DNA marker within the nucleus, the area occupied by the DNA marker within the cell, the total intensity of a cellular protein marker within the cell, the mean intensity of the cellular protein marker within the cell, the mean intensity of the protein marker within the cytoplasm, and the spatial distribution of protein marker within the cells.

7. The method of claim 1, further comprising automatically segmenting the image into individual cells prior to (b).

8. The method of claim 1, further comprising:
(d) extracting a morphological feature of the cells in the image; and
(e) determining the degree to which the morphological feature occurs separately in at least one of live cells and dead cells.

9. The method of claim 1, further comprising:
exposing the population of cells to a stimulus under investigation;
fixing the population of cells; and
marking the at least one cellular component in the population of cells with a marker that is specific for the cellular component after the cells have been exposed to the stimulus.

10. The method of claim 1, wherein automatically using the information about the at least one cellular component to classify individual cells as live or dead comprises applying the information about the cellular component to a mixture model of two Gaussian distributions.

11. A computer program product comprising a machine readable medium on which is provided program instructions for distinguishing live cells from dead cells in a population of cells, the program instructions comprising:
(a) code for providing one or more images of at least one cellular component in a population of cells;
(b) code for analyzing said one or more images to determine, for at least some cells in said population of cells, information about the at least one cellular component; and
(c) code for using the information about the at least one cellular component to classify said at least some cells as live or dead;
wherein the at least one cellular component is selected from cellular protein and DNA.

12. The computer program product of claim 11, wherein the at least one cellular component is DNA and cellular protein.

13. The computer program product of claim 11, wherein the information about the cellular component comprises information about at least one of the total amount, area or distribution of the cellular component in the cell or a region of the cell.

14. The computer program product of claim 11, further comprising code for segmenting the image into individual cells.

15. The computer program product of claim 11, further comprising code for executing the code of (a)-(c) multiple times, each time for a different population of cells, wherein the different populations of cells have been exposed to different levels of a stimulus.

16. A method of distinguishing live cells from dead cells in a population of cells, the method comprising:
(a) providing one or more images of the DNA and protein in a population of cells;
(b) automatically analyzing said one or more images to determine, for at least some cells in said population of cells, information about the DNA and protein; and
(c) automatically applying the information about the DNA and protein classify said at least some cells as live or dead.

17. The method of claim 16, wherein step (b) comprises evaluating an indicator expression for each cell of the at least some cells.

18. The method of claim 17, wherein (c) comprises applying the information to a mixture model of two Gaussian distributions, one for live cells and one for dead cells to classify said at least some cells as live or dead.

19. The method of claim 17, wherein the indicator expression comprises one or more of the following: the standard deviation of the intensity of a DNA marker within the cell, the total intensity of the DNA marker within the nucleus, the number of pixels the DNA marker occupies within the cell, the mean intensity of DNA marker within the nucleus, the total intensity of a cellular protein marker within the cell, the mean intensity of the cellular protein marker within the cell, the mean intensity of the protein marker within the cytoplasm, and the ratio of total or mean intensity of the protein marker within the cytoplasm to those within the nucleus.

20. The method of claim 17, wherein the indicator expression is a combination of least some of the following: the mean intensity of a DNA marker within the cell, the standard deviation of the intensity of the DNA marker within the cell, and the mean intensity of a cellular protein marker within the cell.
